(19) United States
(12) Patent Application Publication
(10) Pub. No.: US 2009/0081176 A1
(43) Pub. Date: Mar. 26, 2009

(54) Cure for the human immunodeficiency virus

(76) Inventor: Rudolph Maravich, La Quinta, CA (US)

Correspondence Address: Rudolph Maravich, 54675 Southern Hills, La Quinta, CA, 92253, US

(21) Appl. No.: 12/231,530

(22) Filed: Sep. 3, 2008

Related U.S. Application Data
(60) Provisional application No. 60/970,798, filed on Sep. 7, 2007.

(51) Int. Cl. A61K 35/14; A61M 1/00

(52) U.S. Cl. 424/93.71; 424/529; 604/22

(57) ABSTRACT

A method whereby a cure is found for the Human Immunodeficiency Virus (HIV) by rebuilding and stimulating the infected individual's immune system.

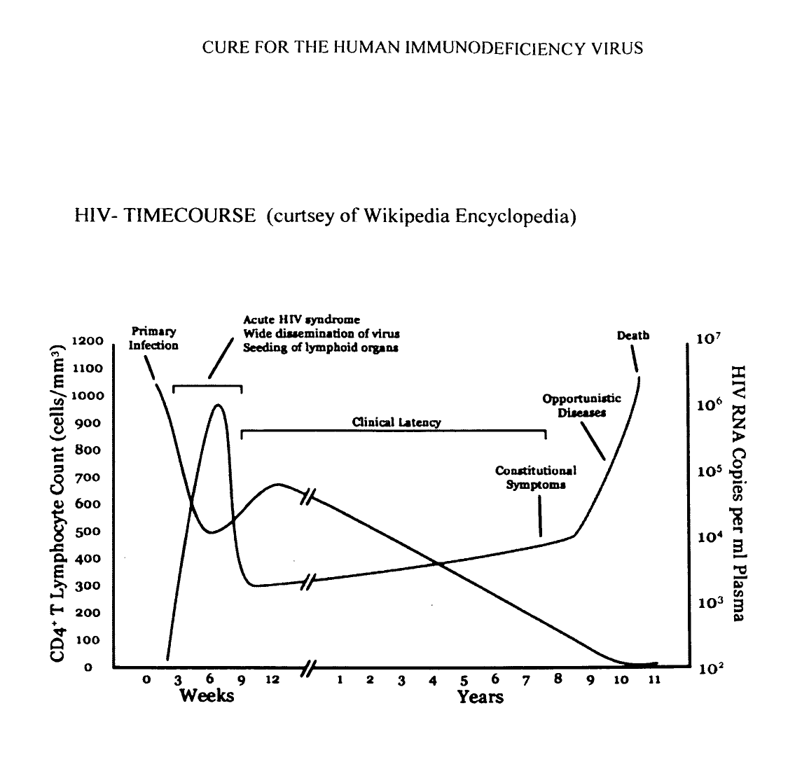

### BACKGROUND OF THE INVENTION

Heretofore, many experiments have been conducted by various independent Pharmaceutical Companies and Medical Research Groups in an attempt to find a cure or a vaccine for the Human Immunodeficiency Virus (HIV). Thus-far all of these experiments have failed to yield a solution to the problem. As recently as September 2007 a major Pharmaceutical Company suspended its trials in its quest to develop a vaccine to prevent AIDS (Acquired Immune Deficiency Syndrome). This attempt was furthest along in development and closely watched by experts in the field. This suspended trial again failed to yield a solution the problem.

With all the past years of research and study, no-one has advanced the study of rebuilding and stimulating the infected individual's immune system through the means of Phlebotomy. The withdrawing of specific amounts of the infected individual's blood so that new fresh replacement blood from the infected individual's own boon marrow will rebuild and stimulate their immune system to combat the virus.

Coupled with but, not necessarily, present day and future days drug medication to help maintain the HIV virus, Phlebotomy as described above may be our answer to the HIV cure.

### SUMMARY OF THE INVENTION

It is the intent of the invention to stimulate and rebuild the infected individual's immune system through Phlebotomy in an attempt to cure the HIV virus. The removal of the infected individual's blood at a given rate and given amount will bring new fresh replacement blood from the infected individual's own bone marrow to rebuild their CD4+T Lymphocyte count to combat and cure the HIV virus.

Within the first year of HIV infection, the CD4+T Lymphocyte count (the virus killer T cells of the immune system) of the infected individual drops from the normal range of 1,050 cells per millimeter cubed of blood to a loss of about half this amount while combating the infection. The HIV RNA copies of infection drop from ten million copies per milliliter of blood Plasma at the onset of the infection to about fifty thousand copies within the same time-frame as the infected individual's immune system tries combat the virus. Without help to the immune system, the HIV RNA copies of infection increase over the years reaching a level of in excess of fifty million copies per milliliter of blood Plasma at which time death may occur. Through Phlebotomy, we should be able to rebuild and restore the CD4+T virus killer cell count back to its near normal level and thus eradicate the virus.

For HIV viral infections that are of a longer nature and prior to receiving early combative help, it will be necessary to establish a cure through Phlebotomy by trial and perhaps with the aid of present day and future day drug medication.

### DESCRIPTION OF THE DRAWING

The drawing is a curtsey of Wikipedia Encyclopedia showing the viewer the Timecourse of the Human Immunodeficiency Virus (HIV). At the left vertical axis is the CD4+T Lymphocyte Count of the infected individual's white blood cells and the right vertical axis is the HIV RNA Copies of the infection.

1. To rebuild and stimulate the infected individual's immune system through Phlebotomy.

2. To rebuild and stimulate the immune system by introducing new blood from the infected individual's own bone marrow.

3. To rebuild and stimulate the immune system by introducing new blood from the infected individual's own bone marrow at a rate of one pint or 450 milliliters every eight weeks or at a rate to be determined.

4. Develop an upward stair-step pattern increasing the infected individual's CD4+T Lymphocyte Count by introducing new blood from the infected individual's own bone marrow at a predetermined rate or as dictated by test results.

5. Target the upward stair-step pattern of CD4+T Lymphocyte Count to 1,000 plus cells per cubic millimeter of Blood Plasma by introducing new blood from the infected individual's own bone marrow at a predetermined rate or as dictated by test results.

6. In a downward stair-step pattern target to reduce the HIV RNA Copies per milliliter of Blood Plasma to less than 1,000 in the infected individual by rebuilding and stimulating the infected individual's immune system by introducing new blood from the infected individual's own bone marrow at a predetermined rate or at a rate dictated by test results.

7. Introduce into the infected individual only White Blood Cells from Donors at a predetermined rate or at a rate dictated by test results in conjunction with items 3, 4, 5, and 6 above.

8. Introduce medication into the infected individual in conjunction with items 3, 4, 5, 6, and 7.

9. In conjunction with items 1 through 8, cure other viruses that invade the Human Body.
